(19) United States
(12) Patent Application Publication
(10) Pub. No.: US 2025/0258825 A1
(43) Pub. Date: Aug. 14, 2025

(54) METHOD, APPARATUS, AND COMPUTER-READABLE MEDIUM FOR RETRIEVAL AUGMENTED GENERATION OF OPTIMAL CODING

(71) Applicant: LateralCare Inc., Southlake, TX (US)

(72) Inventors: Sowri KRISHNAN, Southlake, TX (US); Manikandan NEDUNCHELIAN, Southlake, TX (US)

(21) Appl. No.: 19/049,518

(22) Filed: Feb. 10, 2025

Related U.S. Application Data
(60) Provisional application No. 63/551,702, filed on Feb. 9, 2024.

(51) Int. Cl. G06F 16/2455; G06F 16/22; G16H 10/60

(52) U.S. Cl. CPC G06F 16/2455; G06F 16/2237; G16H 10/60

(57) ABSTRACT

An apparatus, computer-readable medium, and computer-implemented method retrieval augmented generation of optimal coding, including encoding a current claim record comprising codes as a current claim vector, querying a claim vector database to identify claim vectors proximate to the current claim, querying a provision vector database to identify provision vectors corresponding to the codes, applying a predictive large language model (LLM) to the current claim vector, the claim vectors, the provision vectors, and a schedule corresponding to the provisioning structures to generate an optimal coding for the current claim record based on optimization criteria, and transforming the current claim record based at least in part on the determined optimal coding

### RELATED APPLICATION DATA

This application claims priority to U.S. Provisional Application No. 63/551,702, filed Feb. 9, 2024, the disclosure of which is hereby incorporated by reference in its entirety.

### BACKGROUND

Traditionally, the documentation of patient encounters involves recording a plethora of observations and diagnostic details. This process, predominantly manual, is not only labor-intensive but also prone to human error. Such errors can lead to administrative inaccuracies and the potential loss of critical medical insights, which are vital for both patient care and the intricacies of claim processing.

Current systems for claim submission and tracking do not provide any mechanism for viewing historical data in context or adjusting coding to account for previous pattern and behaviors. Furthermore, there are often multiple different coding strategies available for claim submission and there is currently no way to optimize coding for claims that integrates prior claim history, provisions governing coding, and other criteria.

### BRIEF DESCRIPTION OF THE DRAWINGS

FIG. 1 illustrates a flowchart for retrieval augmented generation of optimal coding according to an exemplary embodiment.

FIG. 2 illustrates an example of encoding a current claim record as a current claim vector according to an exemplary embodiment.

FIG. 3 illustrates a flowchart for generating a current claim record according to an exemplary embodiment.

FIG. 4 illustrates a system diagram of a process for generating a current claim record according to an exemplary embodiment.

FIG. 5 illustrates a system chart for determining proximate claim vectors according to an exemplary embodiment.

FIG. 6A illustrates a flowchart for transmitting representations of the current claim vector and the claim vectors proximate to the current claim vector according to an exemplary embodiment.

FIG. 6B illustrates a flowchart for generating and transmitting an approval probability value according to an exemplary embodiment.

FIGS. 7A-7B illustrate a user interface for viewing a current claim vector and proximate claim vectors and an approval probability according to an exemplary embodiment.

FIG. 8 illustrates a flowchart for generating a provision vector database according to an exemplary embodiment.

FIG. 9 illustrates a flowchart for generating a schedule corresponding to the one or more provisioning structures according to an exemplary embodiment.

FIG. 10 illustrates a system chart showing the process for building a provision vector database, extracting a schedule from the provisioning structures, and querying a provision vector database to identify provision vectors corresponding to codes according to an exemplary embodiment.

FIG. 11 illustrates a system chart illustrating the process for generating a transformed current record with the optimal coding according to an exemplary embodiment.

FIG. 12 illustrates the components of the specialized computing environment for retrieval augmented generation of optimal coding according to an exemplary embodiment.

### DETAILED DESCRIPTION

While methods, apparatuses, and computer-readable media are described herein by way of examples and embodiments, those skilled in the art recognize that methods, apparatuses, and computer-readable media for retrieval augmented generation of optimal coding are not limited to the embodiments or drawings described. It should be understood that the drawings and description are not intended to be limited to the particular form disclosed. Rather, the intention is to cover all modifications, equivalents and alternatives falling within the spirit and scope of the appended claims. Any headings used herein are for organizational purposes only and are not meant to limit the scope of the description or the claims. As used herein, the word “may” is used in a permissive sense (i.e., meaning having the potential to) rather than the mandatory sense (i.e., meaning must). Similarly, the words “include,” “including,” and “includes” mean including, but not limited to.

Applicant has discovered a novel method, apparatus, and computer-readable medium for retrieval augmented generation of optimal coding.

The present system integrates two advanced technological systems: an Inference Engine and a Retrieval Augmented Generation (RAG) Engine. Together, these engines revolutionize the processing of documentation and the optimization of coding, significantly alleviating system burdens and markedly enhancing the efficiency of healthcare management systems.

The advanced technological framework disclosed herein utilizes an Inference Engine, implemented as a fine-tuned Large Language Model (LLM), which performs multiple key functions. The Inference Engine interprets and makes sense of the vast array of unstructured text generated by healthcare professionals during patient interactions. This text ranges from doctors' notes to physiotherapists' observations, encompassing a wide spectrum of data that poses a formidable challenge to conventional processing methods. The Inference Engine further optimizes the coding of claim records based on its specialized training and multiple vector databases of prior claims, provisioning structures, and optimization criteria.

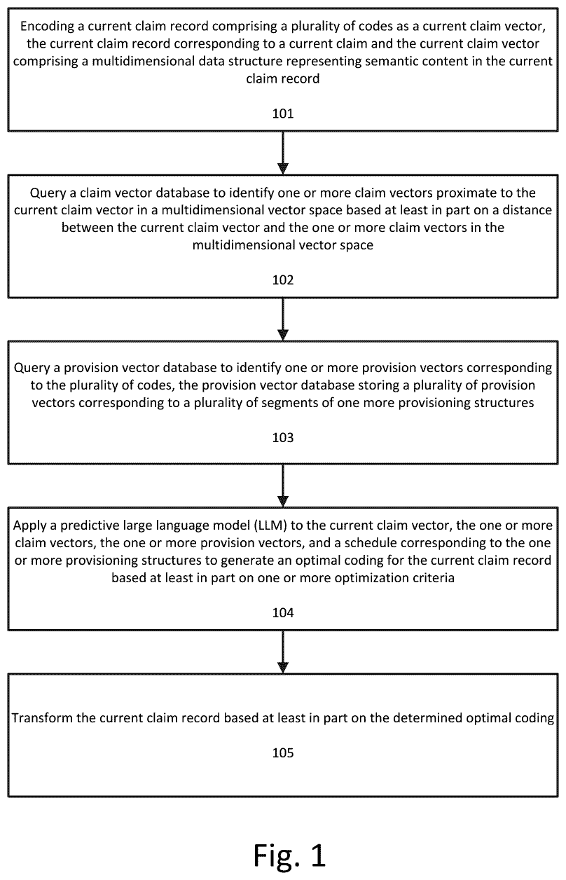

FIG. 1 illustrates a flowchart for retrieval augmented generation of optimal coding according to an exemplary embodiment.

At step 101 a current claim record comprising a plurality of codes is encoded as a current claim vector, the current claim record corresponding to a current claim and the current claim vector comprising a multidimensional data structure representing semantic content in the current claim record.

The current claim record can be a record of a patient visit to a medical provider, such as a doctor, urgent care provider, emergency room, clinic, etc. The plurality of codes can be medical codes, such as ICD (International Classification of Diseases), CPT (Current Procedural Terminology), or HCPCS (Healthcare Common Procedure Coding System) codes.

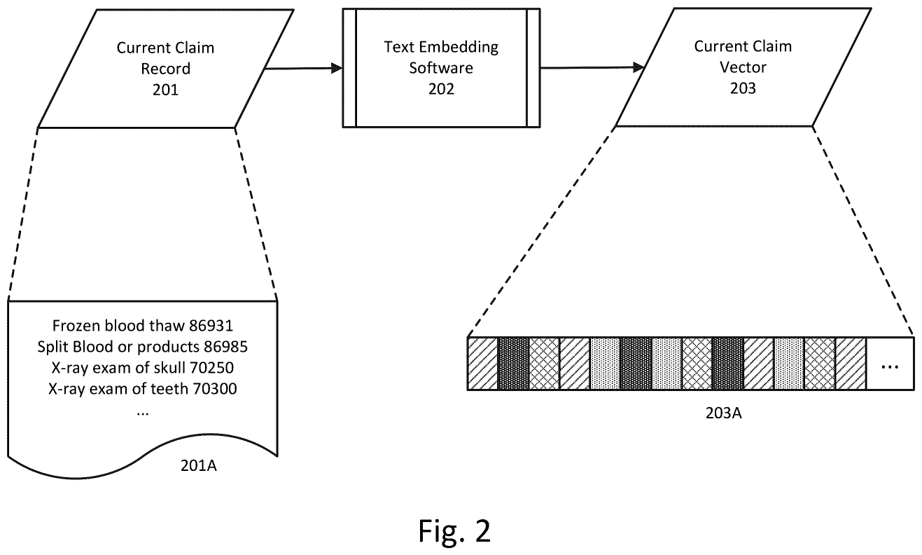

FIG. 2 illustrates an example of encoding a current claim record as a current claim vector according to an exemplary embodiment. As shown in FIG. 2, current claim record 201 is a record of a visit to a medical provider and includes both description unstructured text, as well as medical codes. For example, the record portion 201A includes the description “Frozen blood thaw” and the corresponding code “86931.” The current claim record 201 is provided to text embedding software 202, which converts the current claim record 201 into a current claim vector 203.

Text embedding software 202 projects the text in the current claim record 201 into a high-dimensional latent space, represented as a vector 203. The vector can have any number of dimensions, such as several hundred dimensions or over a thousand dimensions. For example, the vector can have 1536 dimensions or 3072 dimensions. The dimensions can represent different syntactical, semantic, or other attributes, features, or properties of the text that is embedded. Text embedding converts text into a multidimensional data structure storing numerical values that are utilized by large language models (LLMs) for various applications. FIG. 2 illustrates a representation of a portion 203A of the current claim vector. Each dimension is indicated in portion 203A using a different shading pattern, but it is understood that each dimension in the vector stores a numerical value corresponding to the value of that dimension for the vector. For example, the vector can use floating point numbers to store the dimension values for each dimension of the vector.

The codes in the current claim record can be generated by a hospital or medical provider coding department based on the description of services/procedures performed. For example, a medical providers notes can be provided to a coding department, which typically inserts the relevant healthcare codes for billing to a healthcare payer and optionally generates a description of the services provided and procedures performed.

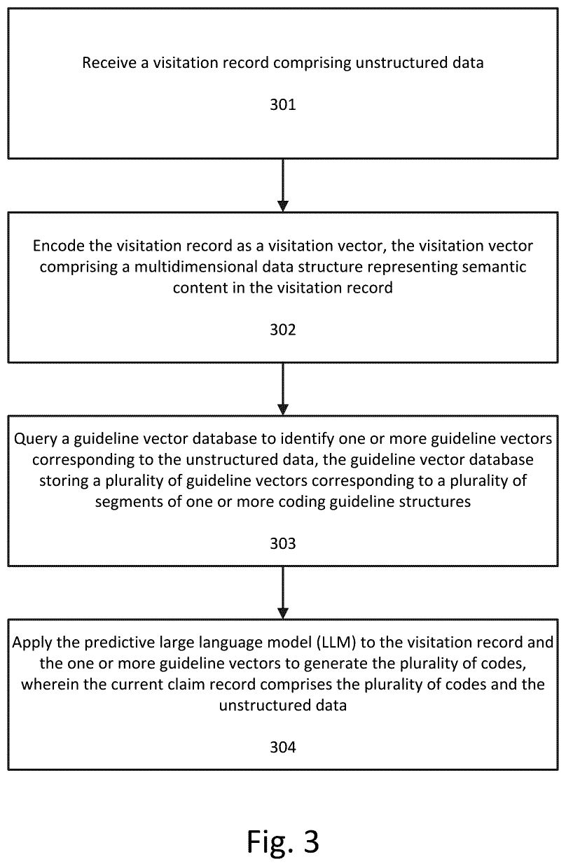

Optionally, the codes in the current claim record can be generated through an inference process using a large language model (LLM). FIG. 3 illustrates a flowchart for generating a current claim record according to an exemplary embodiment.

At step 301 a visitation record comprising unstructured data is received. The visitation record can include healthcare provider notes, diagnostic tests, lab results, or any other documentation of a patient visit. The visitation record can include multiple different documents or notes and can be consolidated or formatted into a standardized format. The visitation record can take any format, such as JSON document.

At step 302 the visitation record is encoded as a visitation vector. The visitation record can be encoded using text embedding software, as discussed previously. The visitation vector is a multidimensional data structure representing semantic content in the visitation record.

At step 303 a guideline vector database is queried to identify one or more guideline vectors corresponding to the unstructured data. The guideline vector database can be queried with the visitation vector to identify guideline vectors corresponding to semantic content that is related to the semantic content of the visitation vector.

The guideline vector database stores a plurality of guideline vectors corresponding to a plurality of segments of one or more coding guideline structures, each guideline vector comprising a multidimensional data structure representing semantic content in a corresponding guideline segment. The one or more coding guideline structures can include healthcare guidelines and documents, such as ICD official guidelines, HCPCS, CPT healthcare coding guidelines, etc. The coding guideline structures can be segmented into segments/chunks prior to being vectorized and stored in the guideline vector database. The guideline vector database can updated in near real-time whenever guidelines change, ensuring that the system always utilizes the most current data.

Step 303 identifies the relevant policies and guidelines in the coding guideline structures that correspond to the procedures, services, or conditions described in the visitation record. For example, if the visitation record includes a certain procedure name, then the visitation vector corresponding to that record will encode a representation of that procedure name. When the visitation vector is used to query the guideline vector database, then vectors can be returned which correspond to sections of the coding guideline structures that reference the procedure.

At step 304 a predictive large language model (LLM) is applied to the visitation record and the one or more guideline vectors to generate the current claim record including the plurality of codes. The LLM is part of the inference engine of the present system and is meticulously fine-tuned for the nuances of the healthcare sector. This model excels in its deep comprehension of complex medical terminologies and its capability to distil these into precise medical codes. These codes are crucial for streamlining billing and claims processes, thereby enhancing the overall efficiency of healthcare administration. The LLM is configured to translate visitation records, such as healthcare professionals notes, into accurate medical codes.

The present system implements a Retrieval Augmented Generation (RAG) pattern by first querying the guideline vector database for vector embeddings that match the visitation record. The returned vector embeddings are then processed by the fine-tuned LLM. This model is adept at deconstructing and reconstructing the text, contextualizing the notes, and predicting the appropriate medical codes (ICD, CPT, HCPCS) based on this analysis.

The predicted codes can optionally be streamed to an application, where medical coders can review and approve them. In instances where the LLM-predicted codes are modified during manual review, this discrepancy can be flagged as a drift or error. The difference can automatically captured and subjected to an automated learning process known as Reinforcement Learning from Human Feedback (RLHF). This process continually refines and enhances the accuracy of the Inference Engine and the LLM.

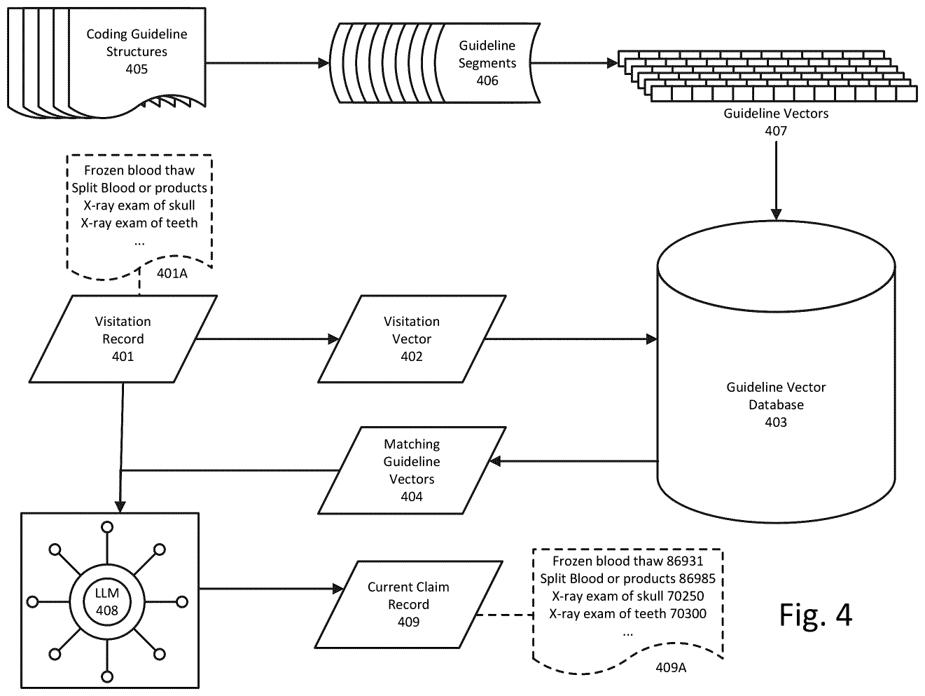

FIG. 4 illustrates a system diagram of a process for generating a current claim record according to an exemplary embodiment. The visitation record 401 can include a description of procedures performed, as shown in note 401A, and is encoded as a visitation vector 402, which is then used to query guideline vector database 403 and return matching guideline vectors 404.

The matching guideline vectors 404 and the visitation record 401 are provided to the fine-tuned LLM 408 (optionally with appropriate prompting, e.g., “generate codes correspond to the visitation record”) to generate the current claim record 409. As shown in record portion 409A, the current claim record includes medical codes corresponding to the procedures in the visitation record.

As shown in FIG. 4, the guideline vector database 403 is generated using the coding guideline structures 405. The coding guideline structures 405 are divided into guideline segments 406 and then encoded as guideline vectors 407. The segmentation of the coding guideline structures 405 can be optimized based on the type of guideline structure, such that different types of files can be segmented differently. For example, PDF files can be segmented into 1-page chunks, whereas Word documents can be segmented differently. The guideline vectors 407 are then stored in the guideline vector database 403.

Returning to FIG. 1, regardless of how the current claim record is generated, at step 102 a claim vector database is queried to identify one or more claim vectors proximate to the current claim vector in a multidimensional vector space based at least in part on a distance between the current claim vector and the one or more claim vectors in the multidimensional vector space. The claim vector database stores a plurality of claim vectors corresponding to a plurality of prior claims, each claim vector comprising a multidimensional data structure representing semantic content in a corresponding prior claim. The vector space of the claim vectors can have many dimensions, including dimensions corresponding to charges, diagnosis codes, diagnosis related group codes, procedure codes, revenue codes, etc.

Proximity can be determined in a variety of ways, such as cosine similarity or distance. Proximity can also be determined based on clustering of the vectors, with vectors within the same cluster being considered proximate to the current claim vector. A variety of techniques can be used for clustering. For example, the density-based spatial clustering of applications with noise (DBSCAN) clustering method can be used. Clustering can also be performed using the Balanced Iterative Reducing and Clustering using Hierarchies (“BIRCH”) method. Additionally, clustering methods other than DBSCAN or BIRCH can be used during the clustering step. For example, clustering algorithms such as K-means or DENGRIS can be used to group the vectors into clusters.

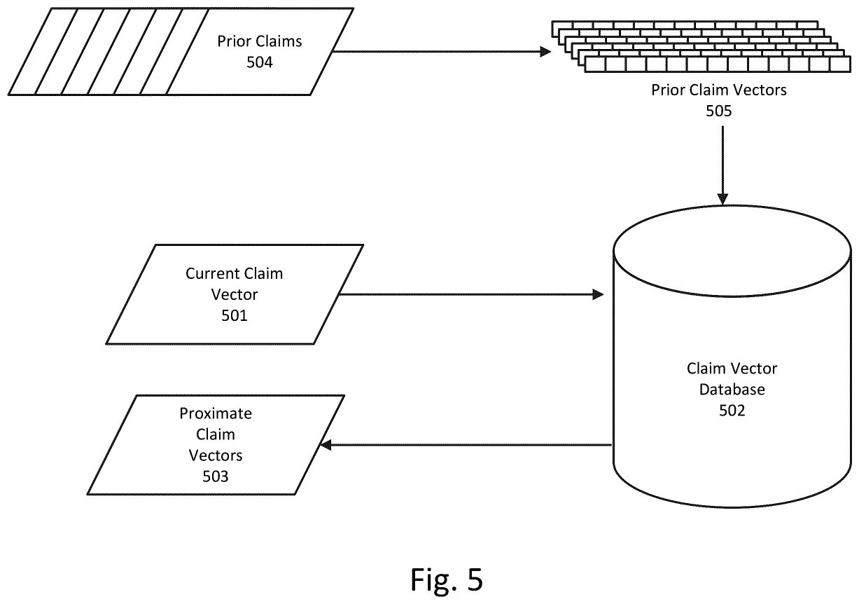

FIG. 5 illustrates a system chart for determining proximate claim vectors according to an exemplary embodiment. Prior claims 504 are encoded/vectorized to generate prior claim vectors 505. The encoding can be performed through text embedding software, as discussed previously. The current claim vector 501 is then used to query the claim vector database 502 to retrieve the proximate claim vectors 503.

Once the proximate claim vectors are retrieved, a user can optionally be presented with a graphical user interface (GUI) showing the current claim record and the proximate claim vectors. In the complex world of healthcare management, providers often face the challenge of not being able to effectively visualize and compare claim data. This limitation hinders their ability to identify deviations and drifts across various dimensions such as codes, contracts, and patient notes. Understanding these nuances is crucial for optimizing claim management and revenue cycle processes. The present system addresses this critical issue by transforming the vector space associated with prior claim vectors. The process involves scaling down the dimensions of these vectors from a large number of dimensions, such as 1536, to a more manageable three or two. This dimensionality reduction is key to enabling the visualization of these vectors in a 3D or 2D graph. Such a graphical representation is not just a visual aid; it's a powerful tool that allows users to interact with and analyze the data in an intuitive and insightful manner.

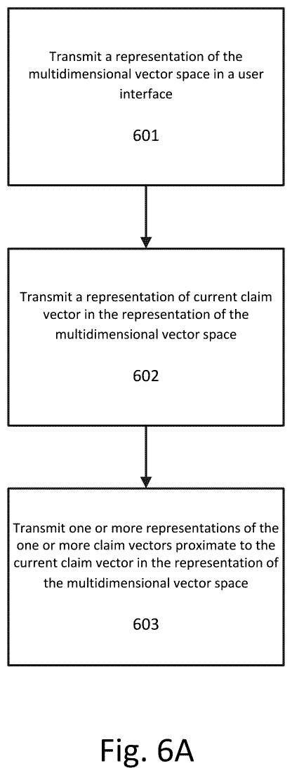

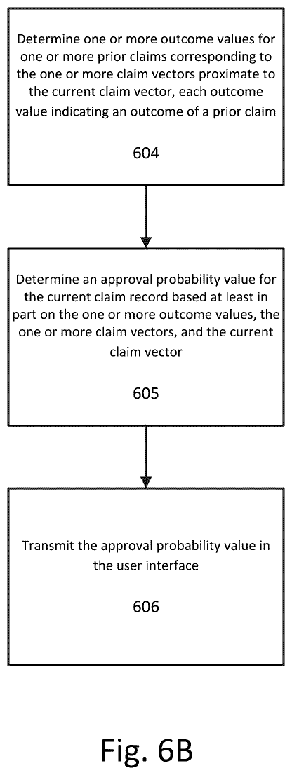

FIG. 6A illustrates a flowchart for transmitting representations of the current claim vector and the claim vectors proximate to the current claim vector according to an exemplary embodiment.

At step 601 a representation of the multidimensional vector space is transmitted in a user interface. The representation of multidimensional vector space can be generated by reducing the number of dimensions in the vector space using a variety of dimensionality reduction techniques. One dimensionality reduction technique that can be used is the Principal Components Analysis (“PCA”) method which reduces the number of the data object's dimensions as compared to the number of data object's dimensions in the original universe of discourse. The PCA input data dimensionality reduction method transforms input data coordinates in such way that eigenvectors of the covariance matrix become new coordinate axis. Horn's Parallel Analysis (“PA”) technique can be used with PCA. PA is based on comparing eigenvalues of an actual data set with eigenvalues of an artificial data set of uncorrelated normal variables of the same dimensionality as the actual data set. Of course, techniques other than the combination of the PCA and PA methods can be used to reduce data dimensionality of the training data. For example, the Linear Discriminant Analysis method or the Sufficient Dimensionality Reduction approach can also be used to achieve the objective of reducing dimensionality. Another technique for dimensionality reduction that can be utilized is t-SNE(t-Distributed Stochastic Neighbor Embedding).

At step 602 a representation of current claim vector is transmitted in the representation of the multidimensional vector space. Once again, the above-mentioned dimensionality reduction techniques can be used to represent the current claim vector in two or three dimensions.

At step 603 one or more representations of the one or more claim vectors proximate to the current claim vector are transmitted in the representation of the multidimensional vector space. These vectors can also be mapped to the reduced dimensions using the above-mentioned dimensionality reduction techniques.

In addition to the representation of the current claim vector and the proximate vectors, the present system can determine and present relevant information about the proximate claim vectors (such as whether the prior claims were approved or rejected), and a probability of success of the current claim record. FIG. 6B illustrates a flowchart for generating and transmitting an approval probability value according to an exemplary embodiment. The steps shown in FIG. 6B can be performed in conjunction with, after, or instead of the steps shown in FIG. 6A.

At step 604 one or more outcome values are determined for one or more prior claims corresponding to the one or more claim vectors proximate to the current claim vector, each outcome value indicating an outcome of a prior claim. The outcome values can indicate the outcomes of the prior claims, such as whether the claim was approved or rejected, or additional outcome values, such as the time/delay required for approval or rejection, or other outcomes. The outcome values can be determined by querying a database outside the vector database that stores the prior claims and metadata about prior claims, such as outcomes.

At step 605 an approval probability value is determined for the current claim record based at least in part on the one or more outcome values, the one or more proximate claim vectors, and the current claim vector. The approval probability value can be determined on a variety of factors, such as whether the proximate claim vectors were approved, how many proximate claim vectors were approved, and the relative distances between the current claim vector and proximate claim vectors that were approved or rejected.

At step 606 the approval probability value is transmitted in the user interface. The approval probability can be shown in a variety of ways, such as a numerical percentage, a color code with high probabilities (e.g., greater than 70%) shown in green, low probabilities (e.g., less than 50%) in red, and medium probabilities (e.g., between 50%-70%) shown in yellow. Of course, these indicators are provided only as an example, and the approval probabilities can be displayed in a variety of ways.

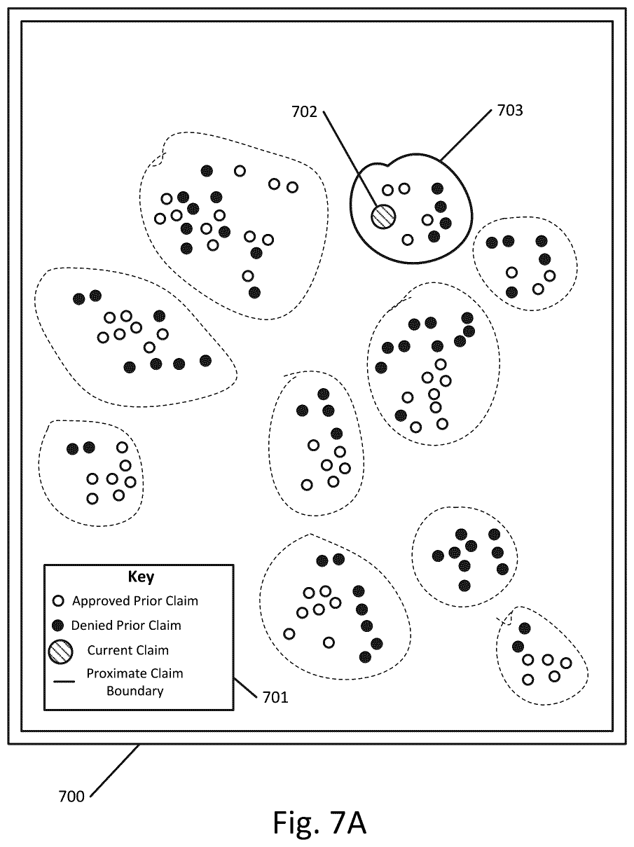

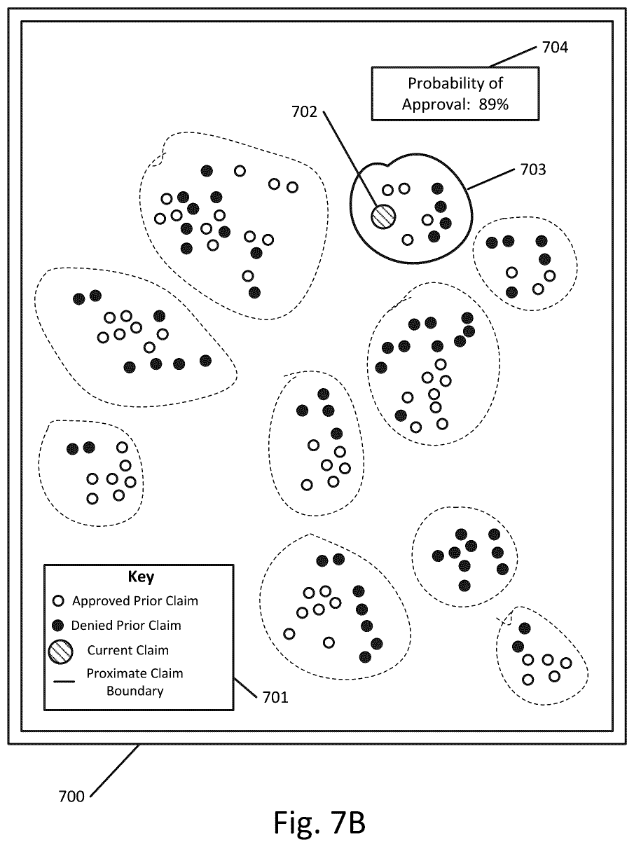

FIGS. 7A-7B illustrate a user interface for viewing a current claim vector and proximate claim vectors and an approval probability according to an exemplary embodiment. FIG. 7A illustrates a user interface 700 showing a two-dimensional vector space, which can be generated from the multidimensional vector space using the dimensionality reduction techniques described above. Of course, a two-dimensional space is presented for case of illustration only, and it is understood that a three-dimensional space vector space can be utilized as well.

User interface 700 includes a key 701 explaining the icons shown in the user interface. As shown in the key 701, the claim vectors corresponding to prior claims that were rejected are shown as black circles and the claim vectors corresponding to prior claims that were approved are shown as white circles. Additionally, the current claim vector 702 is shown with a larger circle having a diagonal line pattern. The dashed lines indicate groupings of claim vectors that are close to one another in the vector space. The solid lines 704 indicate a proximate claim vector boundary of the proximate claim vectors close to the current claim vector. A predetermined distance threshold can be used to determine the proximate claim vector boundary in order to classify prior claim vectors as proximate. The distance threshold can also be automatically computed based on clustering algorithms that cluster the vectors.

FIG. 7B illustrates the user interface 700 with an indication of the probability of approval 704. As shown in user interface element 704, the probability of approval is 89%. As discussed previously, this probability can be determined based on the closest proximate vectors within boundary 703.

The graph and user interface serves as an interactive platform where users can observe clusters of prior claims/visits. They can engage with individual vector chunks or select groups to examine all the attributes within a chosen segment. This interactive capability is pivotal in unravelling the complexities of claim data, offering a clear and comprehensive view of how different claims are related or divergent. This visualization and interaction with the data helps users to dissect various aspects of a claim. For instance, they can investigate why a particular visit deviated from its expected cluster. Such insights are invaluable in identifying anomalies and patterns that might otherwise go unnoticed.

This approach facilitates the identification of potential new claiming strategies. By analyzing the proximity of visits in the vector space and examining the differences in their attributes, such as the order of diagnosis or treatment codes, users can uncover opportunities for optimizing claims. For example, if two visits are closely related but have different arrangements of diagnoses that lead to a higher claim amount for one, this insight can be pivotal. Users can use this information to advise the coding team about specific drifts that could result in optimized revenue for similar claims.

The present system enables the more efficient handling of denials/rejections. As discussed above, the denied claims are converted into text embedding vectors and projected into a 2D or 3D vector space view. The denied claim can pass through two vector indexes and based on its occupying neighbors, a confidence score can be calculated and the insights can be presented to the user. The two vector indexes can be the Claims Parity Index (CPI) and Denials Parity Index (DPI).

The Claims Parity Index (CPI) functions as a three-dimensional vector visualization tool. It is intricately connected to all successful claim submissions historically associated with a particular provider/payer. When a denied claim is introduced into this index's vector space, it is represented as a vector. Due to the nature of this data representation, the denied claim vector tends to be positioned in proximity to other vectors that correspond to historically successful claims. The system then employs a method to measure the cosine distance between the vector of the denied claim and those of successful claims. Based on this distance, one or more vectors representing successful claims are selected and fed into a custom AI model. This model is adept at making precise predictions about which data points need to be altered in the denied claim to align it more closely with the characteristics of a successful claim.

The Denials Parity Index (DPI) operates on a similar principle to the CPI but with a focus on denied claims. The vector index in this case is populated with vectors representing past denied claims. This setup allows for a comprehensive visualization of the current denied claim in the context of historical denial patterns. By analyzing how the current claim compares to these past denials, the system can identify recurring patterns or anomalies in the denial process, providing valuable insights for addressing and rectifying these issues.

The CPI and DPI work in tandem to enhance the denial management process. By leveraging these two indices, the present system provides a more nuanced and data-driven approach to handling denials. This method not only increases the efficiency of the process but also contributes to a higher success rate in converting denied claims into successful ones. The integration of these advanced analytical tools provides significant advantages to systems for optimizing coding.

Optionally, the process described in FIGS. 6A-6B and shown in FIGS. 7A-7B can be performed after transformation of current claim record (i.e., step 105 of FIG. 1), discussed below. In this case, the user is able to see the probability of approval of the transformed current claim record and compare it with the probability prior to transformation.

Returning to FIG. 1, at step 103 a provision vector database is queried to identify one or more provision vectors corresponding to the plurality of codes. The provision vector database stores a plurality of provision vectors corresponding to a plurality of segments of one more provisioning structures, each provision vector comprising a multidimensional data structure representing semantic content in a corresponding segment.

The provisioning structures can be segmented into segments/chunks prior to being encoded/vectorized and stored in the provision vector database. In this phase, the processed content is transformed into text embeddings using Large Language Models (LLMs). The content is segmented into manageable chunks, which are then vectorized and stored in a vector database. This database becomes a valuable resource for specialists, enabling them to conduct ad-hoc analyses as needed. The vectorization of provisioning structures not only simplifies the retrieval process but also enhances the precision and speed of analysis. Optionally, the system can include a chat functionality with a Generative Pre-trained Transformer (GPT) and the LLM that enables users to query the provisioning structures to obtain accurate information about provisions in the provisioning structure.

The one or more provisioning structures can include contracts, such as health insurance contracts, payor contracts, or other documents specifying the terms and conditions pertaining to different codes. For example, the provisioning structure can be a contract between a hospital and health insurance company that specifies the terms and conditions, and payment amounts for different medical procedures and codes. In the intricate and demanding sphere of Revenue Cycle Management (RCM), one of the most formidable challenges is the interpretation and application of complex payer or insurance contracts. These contracts are crucial for accurately filing claims based on various codes and stipulations.

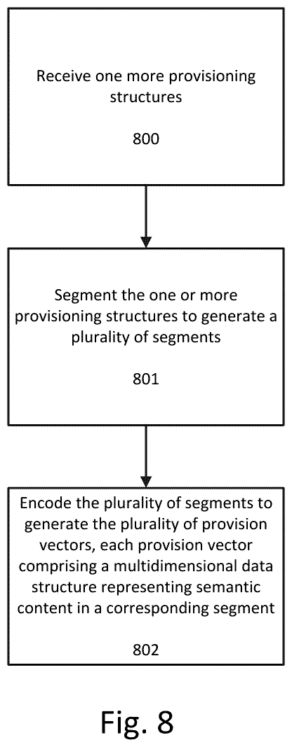

FIG. 8 illustrates a flowchart for generating a provision vector database according to an exemplary embodiment. At step 801 one more provisioning structures are received. As discussed above, the provisioning structures can be complex payer or insurance contracts. These contracts are crucial for accurately filing claims based on various codes and stipulations.

Optionally, after receiving the provisioning structures, the provisioning structures can be sanitized to ensure data quality. The sanitation process can include correcting for errors or data quality issues, such incorrectly formatted or missing characters or sections of the documents. The sanitation process can also include standardizing and formatting the provisioning structures into a standardized format.

At step 802 the one or more provisioning structures are segmented to generate a plurality of segments. The provisioning structures can be segmented according to various criteria, such as the file type, the type of provisioning structure (e.g., contract type), or other criteria. The segments/chunks can be based on page breaks, a predetermined number of words or characters, or larger chunks such as different sections or clauses of a contract.

At step 803 the plurality of segments are encoded to generate the plurality of provision vectors, each provision vector comprising a multidimensional data structure representing semantic content in a corresponding segment. The encoding process can be performed with text embedding software as discussed above. The vector space of the provision vectors can have many dimensions, including dimensions corresponding to payer names, payer codes, financial classes, etc.

Along with segmenting and encoding the provisioning structures as vectors, the present system can extract additional information from the provisioning structures that are relevant to claim optimization process. This additional information includes a schedule of fees/costs pertaining to different codes, which can be utilized by the LLM as part of the optimization process.

In this step, the system automatically compiles a comprehensive table summarizing payment codes and their corresponding rates. This summary can optionally be reviewed by an RCM specialist for validation and accuracy and then stored in a database. The Fee Schedule can be used in conjunction with the codes generated by the Inference Engine, facilitating a seamless crosswalk and ensuring that claims are filed accurately and in accordance with the specific stipulations of each contract.

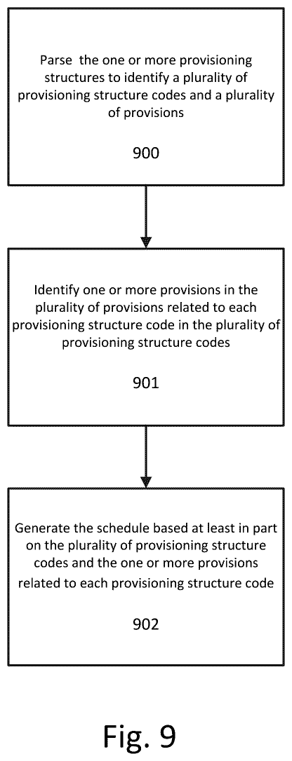

FIG. 9 illustrates a flowchart for generating a schedule corresponding to the one or more provisioning structures according to an exemplary embodiment.

At step 901 the one or more provisioning structures are parsed to identify a plurality of provisioning structure codes and a plurality of provisions. The provisioning structure codes can correspond to healthcare/medical codes and the provisions can be unstructured data or text. The provisioning structure codes can be identified using a variety of natural language processing techniques, regular expression matching, and/or lexical analysis. The provisions can be identified based on proximity/distance to provisioning structure codes.

At step 902 one or more provisions in the plurality of provisions related to each provisioning structure code in the plurality of provisioning structure codes are identified. The related provisions can be identified based on proximity/distance to each provisioning structure code. For example, a provisioning structure code can first be identified using regular expression matching and a proximity search can identify provisions on the same page or within a certain number of characters.

At step 903 the schedule is generated based at least in part on the plurality of provisioning structure codes and the one or more provisions related to each provisioning structure code. The schedule includes provisioning structure codes and corresponding fees or amounts. This step can utilize pre-existing knowledge about the layout and format of the provisioning structures, probabilistic methods for determining fees, or other techniques for matching a provisioning structure code to an amount. The final output of this step is a fee schedule with codes and corresponding amounts.

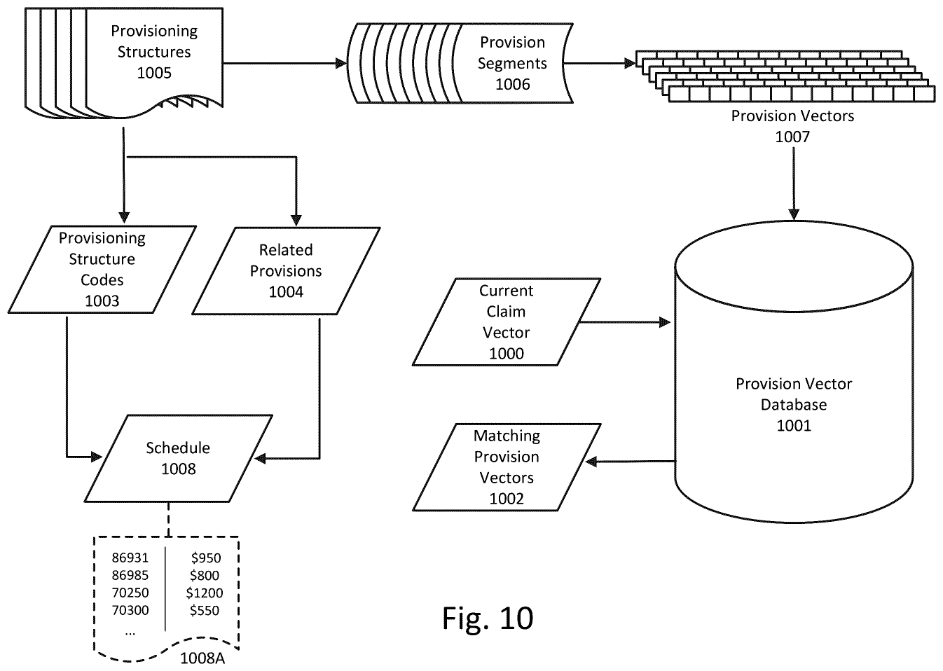

FIG. 10 illustrates a system chart showing the process for building a provision vector database, extracting a schedule from the provisioning structures, and querying a provision vector database to identify provision vectors corresponding to codes according to an exemplary embodiment.

The provisioning structures 1005 are divided into provision segments 1006. These segments 1006 are encoded to generate the provision vectors 1007, which are then stored in the provision vector database 1001. As shown in the FIG. 10, the provisioning structures are parsed as discussed above to extract provisioning structure codes 1003 and related provisions 1004. The provisioning structure codes 1003 and related provisions 1004 are then used to generate the schedule 1008. The portion 1008A of schedule 1008 illustrates an example of the format of the schedule, including codes and corresponding amounts/fees. As additionally shown in FIG. 10, the current claim vector 1000 is used to query the provision vector database 1001, which returns matching provision vectors 1002.

Returning to FIG. 1, at step 104 the predictive large language model (LLM) is applied to the current claim vector, the one or more claim vectors, the one or more provision vectors, and the schedule corresponding to the one or more provisioning structures to generate an optimal coding for the current claim record based at least in part on one or more optimization criteria.

The optimization criteria can include one or more of a predicted probability of approval of the current claim record, an overall revenue resulting from the current claim record, a predicted response time for the current claim record, reordering to match payer expectation. Multiple sets of optimization criteria can be utilized as well. For example, the LLM can be configured to first optimize for probability of approval and then optimize according to maximum revenue. The optimization process can optimize along different criteria in parallel (e.g., revenue and approval probability) and present both options to users for selection. Options can be presented with projected review and risk score/probability of success to aid users in selection of an optimization strategy.

The generated optimal coding can include the optimal codes recommended for utilization in the current claim record. The generated optimal coding can also include one or more transformations to be applied to the current claim record to transform the record into one having the optimal coding.

At step 105 the current claim record is transformed based at least in part on the determined optimal coding. The step of transforming the current claim record can be performed in a variety of ways, such as:
replacing at least one code in the plurality of codes with at least one alternate code;
removing at least one code in the plurality of codes;
adding at least one new code to the plurality of codes;
changing an ordering of two or more codes in the plurality of codes; and/or
modifying a description associated with at least one code in the plurality of codes.

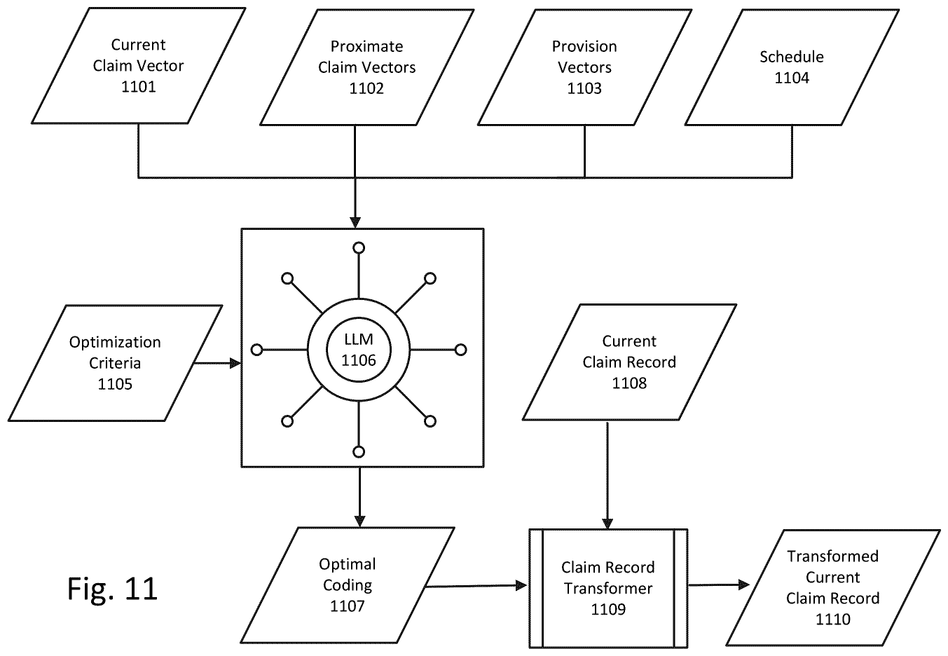

FIG. 11 illustrates a system chart illustrating the process for generating a transformed current record with the optimal coding according to an exemplary embodiment.

As shown in FIG. 11, the current claim vector 1101, the proximate claim vector 1102, the provision vectors 1103, and the schedule 1104 are all provided as input to the LLM 1106, along with optimization criteria 1105. The LLM generates an optimal coding 1107 based on all the inputs, and this optimal coding 1107 is applied to the current claim record 1108 by a claim record transformer 1109 to produce a transformed current claim record 1110.

Optionally, this transformed current claim record can be encoded as a vector and viewed in a user interface, along with proximate vectors and a probability of approval, as shown in FIGS. 6A-6B and 7A-7B. This allows users of the system to evaluate the transformed current claim and make any additional necessary changes.

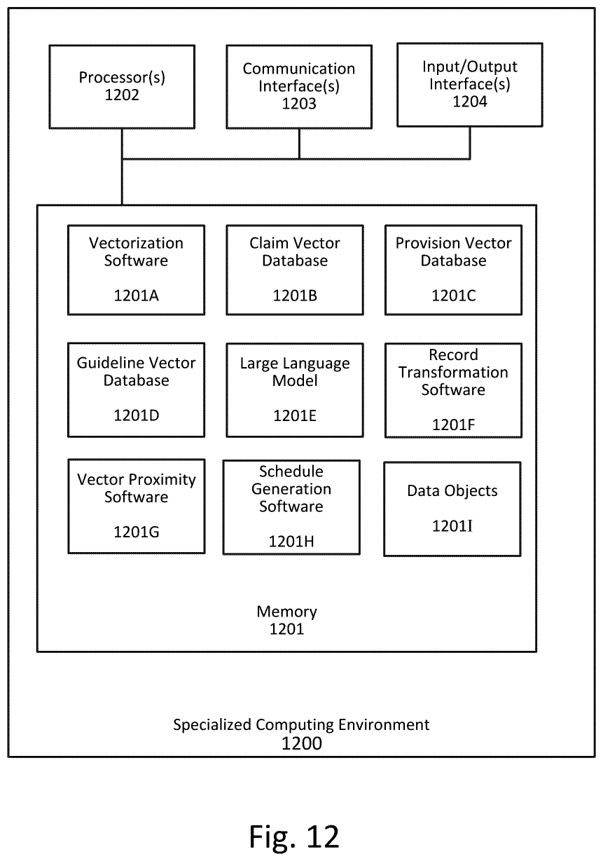

FIG. 12 illustrates the components of the specialized computing environment 1200 configured to perform the processes described herein. Specialized computing environment 1200 is a computing device that includes a memory 1201 that is a non-transitory computer-readable medium and can be volatile memory (e.g., registers, cache, RAM), non-volatile memory (e.g., ROM, EEPROM, flash memory, etc.), or some combination of the two.

As shown in FIG. 12, memory 1201 can include vectorization/encoding/text embedding software 1201A, a claim vector database 1201B, a provision vector database 1201C, a guideline vector database 1201D, a large language model 1201E, record transformation software 1201F, vector proximity software 1201G, schedule generation software 1201H, and one or more data objects 12011. Each of the software components in memory 1201 store specialized instructions and data structures configured to perform the corresponding functionality and techniques described herein.

All of the software stored within memory 1201 can be stored as a computer-readable instructions, that when executed by one or more processors 1202, cause the processors to perform the functionality described with respect to FIGS. 1-11.

Processor(s) 1202 execute computer-executable instructions and can be a real or virtual processors. In a multi-processing system, multiple processors or multicore processors can be used to execute computer-executable instructions to increase processing power and/or to execute certain software in parallel.

Specialized computing environment 1200 additionally includes a communication interface 903, such as a network interface, which is used to communicate with devices, applications, or processes on a computer network or computing system, collect data from devices on a network, and implement encryption/decryption actions on network communications within the computer network or on data stored in databases of the computer network. The communication interface conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Specialized computing environment 1200 further includes input and output interfaces 1204 that allow users (such as system administrators) to provide input to the system to display information, to edit data stored in memory 1201, or to perform other administrative functions.

An interconnection mechanism (shown as a solid line in FIG. 12), such as a bus, controller, or network interconnects the components of the specialized computing environment 1200.

Input and output interfaces 1204 can be coupled to input and output devices. For example, Universal Serial Bus (USB) ports can allow for the connection of a keyboard, mouse, pen, trackball, touch screen, or game controller, a voice input device, a scanning device, a digital camera, remote control, or another device that provides input to the specialized computing environment 1200.

Specialized computing environment 1200 can additionally utilize a removable or non-removable storage, such as magnetic disks, magnetic tapes or cassettes, CD-ROMs, CD-RWs, DVDs, USB drives, or any other medium which can be used to store information and which can be accessed within the specialized computing environment 1200.

Having described and illustrated the principles of our invention with reference to the described embodiment, it will be recognized that the described embodiment can be modified in arrangement and detail without departing from such principles. It should be understood that the programs, processes, or methods described herein are not related or limited to any particular type of computing environment, unless indicated otherwise. Various types of general purpose or specialized computing environments may be used with or perform operations in accordance with the teachings described herein. Elements of the described embodiment shown in software may be implemented in hardware and vice versa.

In view of the many possible embodiments to which the principles of our invention may be applied, we claim as our invention all such embodiments as may come within the scope and spirit of the following claims and equivalents thereto.

What is claimed is:

1. A method executed by one or more computing devices for retrieval augmented generation of optimal coding, the method comprising:
encoding a current claim record comprising a plurality of codes as a current claim vector, the current claim record corresponding to a current claim and the current claim vector comprising a multidimensional data structure representing semantic content in the current claim record;
querying a claim vector database to identify one or more claim vectors proximate to the current claim vector in a multidimensional vector space based at least in part on a distance between the current claim vector and the one or more claim vectors in the multidimensional vector space, the claim vector database storing a plurality of claim vectors corresponding to a plurality of prior claims, each claim vector comprising a multidimensional data structure representing semantic content in a corresponding prior claim;
querying a provision vector database to identify one or more provision vectors corresponding to the plurality of codes, the provision vector database storing a plurality of provision vectors corresponding to a plurality of segments of one more provisioning structures, each provision vector comprising a multidimensional data structure representing semantic content in a corresponding segment;
applying a predictive large language model (LLM) to the current claim vector, the one or more claim vectors, the one or more provision vectors, and a schedule corresponding to the one or more provisioning structures to generate an optimal coding for the current claim record based at least in part on one or more optimization criteria; and
transforming the current claim record based at least in part on the determined optimal coding.

2. The method of claim 1, wherein the current claim record is generated by:
receiving a visitation record comprising unstructured data;
encoding the visitation record as a visitation vector, the visitation vector comprising a multidimensional data structure representing semantic content in the visitation record;
querying a guideline vector database to identify one or more guideline vectors corresponding to the unstructured data, the guideline vector database storing a plurality of guideline vectors corresponding to a plurality of segments of one or more coding guideline structures, each guideline vector comprising a multidimensional data structure representing semantic content in a corresponding segment; and
applying the predictive large language model (LLM) to the visitation record and the one or more guideline vectors to generate the current claim record including the plurality of codes.

3. The method of claim 1, further comprising:
transmitting a representation of the multidimensional vector space in a user interface;
transmitting a representation of current claim vector in the representation of the multidimensional vector space; and
transmitting one or more representations of the one or more claim vectors proximate to the current claim vector in the representation of the multidimensional vector space.

4. The method of claim 3, further comprising:
determining one or more outcome values for one or more prior claims corresponding to the one or more claim vectors proximate to the current claim vector, each outcome value indicating an outcome of a prior claim;
determining an approval probability value for the current claim record based at least in part on the one or more outcome values, the one or more claim vectors, and the current claim vector; and
transmitting the approval probability value in the user interface.

5. The method of claim 1, wherein the provision vector database is generated by:
receiving one more provisioning structures;
segmenting the one or more provisioning structures to generate a plurality of segments; and
encoding the plurality of segments to generate the plurality of provision vectors, each provision vector comprising a multidimensional data structure representing semantic content in a corresponding segment.

6. The method of claim 5, wherein the schedule corresponding to the one or more provisioning structures is generated by:
parsing the one or more provisioning structures to identify a plurality of provisioning structure codes and a plurality of provisions;
identifying one or more provisions in the plurality of provisions related to each provisioning structure code in the plurality of provisioning structure codes; and
generating the schedule based at least in part on the plurality of provisioning structure codes and the one or more provisions related to each provisioning structure code.

7. The method of claim 1, wherein the optimization criteria comprises one or more of:
a predicted probability of approval of the current claim record;
an overall revenue resulting from the current claim record; or
a predicted response time for the current claim record.

8. The method of claim 1, wherein transforming the current claim record based at least in part on the determined optimal coding comprises one or more of:
replacing at least one code in the plurality of codes with at least one alternate code;
removing at least one code in the plurality of codes;
adding at least one new code to the plurality of codes;
changing an ordering of two or more codes in the plurality of codes; or
modifying a description associated with at least one code in the plurality of codes.

9. An apparatus for retrieval augmented generation of optimal coding, the apparatus comprising:
one or more processors; and
one or more memories operatively coupled to at least one of the one or more processors and having instructions stored thereon that, when executed by at least one of the one or more processors, cause at least one of the one or more processors to:
encode a current claim record comprising a plurality of codes as a current claim vector, the current claim record corresponding to a current claim and the current claim vector comprising a multidimensional data structure representing semantic content in the current claim record;
query a claim vector database to identify one or more claim vectors proximate to the current claim vector in a multidimensional vector space based at least in part on a distance between the current claim vector and the one or more claim vectors in the multidimensional vector space, the claim vector database storing a plurality of claim vectors corresponding to a plurality of prior claims, each claim vector comprising a multidimensional data structure representing semantic content in a corresponding prior claim;
query a provision vector database to identify one or more provision vectors corresponding to the plurality of codes, the provision vector database storing a plurality of provision vectors corresponding to a plurality of segments of one more provisioning structures, each provision vector comprising a multidimensional data structure representing semantic content in a corresponding segment;
apply a predictive large language model (LLM) to the current claim vector, the one or more claim vectors, the one or more provision vectors, and a schedule corresponding to the one or more provisioning structures to generate an optimal coding for the current claim record based at least in part on one or more optimization criteria; and
transform the current claim record based at least in part on the determined optimal coding.

10. The apparatus of claim 9, wherein the current claim record is generated by:
receiving a visitation record comprising unstructured data;
encoding the visitation record as a visitation vector, the visitation vector comprising a multidimensional data structure representing semantic content in the visitation record;
querying a guideline vector database to identify one or more guideline vectors corresponding to the unstructured data, the guideline vector database storing a plurality of guideline vectors corresponding to a plurality of segments of one or more coding guideline structures, each guideline vector comprising a multidimensional data structure representing semantic content in a corresponding segment; and
applying the predictive large language model (LLM) to the visitation record and the one or more guideline vectors to generate the current claim record including the plurality of codes.

11. The apparatus of claim 9, wherein at least one of the one or more memories has further instructions stored thereon that, when executed by at least one of the one or more processors, cause at least one of the one or more processors to:
transmit a representation of the multidimensional vector space in a user interface;
transmit a representation of current claim vector in the representation of the multidimensional vector space; and
transmit one or more representations of the one or more claim vectors proximate to the current claim vector in the representation of the multidimensional vector space.

12. The apparatus of claim 11, wherein at least one of the one or more memories has further instructions stored thereon that, when executed by at least one of the one or more processors, cause at least one of the one or more processors to:
determine one or more outcome values for one or more prior claims corresponding to the one or more claim vectors proximate to the current claim vector, each outcome value indicating an outcome of a prior claim;
determine an approval probability value for the current claim record based at least in part on the one or more outcome values, the one or more claim vectors, and the current claim vector; and
transmit the approval probability value in the user interface.

13. The apparatus of claim 9, wherein the provision vector database is generated by:
receiving one more provisioning structures;
segmenting the one or more provisioning structures to generate a plurality of segments; and
encoding the plurality of segments to generate the plurality of provision vectors, each provision vector comprising a multidimensional data structure representing semantic content in a corresponding segment.

14. The apparatus of claim 13, wherein the schedule corresponding to the one or more provisioning structures is generated by:
parsing the one or more provisioning structures to identify a plurality of provisioning structure codes and a plurality of provisions;
identifying one or more provisions in the plurality of provisions related to each provisioning structure code in the plurality of provisioning structure codes; and
generating the schedule based at least in part on the plurality of provisioning structure codes and the one or more provisions related to each provisioning structure code.

15. The apparatus of claim 9, wherein the optimization criteria comprises one or more of:
a predicted probability of approval of the current claim record;
an overall revenue resulting from the current claim record; or
a predicted response time for the current claim record.

16. The apparatus of claim 9, wherein transforming the current claim record based at least in part on the determined optimal coding comprises one or more of:
replacing at least one code in the plurality of codes with at least one alternate code;
removing at least one code in the plurality of codes;
adding at least one new code to the plurality of codes;
changing an ordering of two or more codes in the plurality of codes; or
modifying a description associated with at least one code in the plurality of codes.

17. At least one non-transitory computer-readable medium storing computer-readable instructions for retrieval augmented generation of optimal coding that, when executed by one or more computing devices, cause at least one of the one or more computing devices to:
encode a current claim record comprising a plurality of codes as a current claim vector, the current claim record corresponding to a current claim and the current claim vector comprising a multidimensional data structure representing semantic content in the current claim record;
query a claim vector database to identify one or more claim vectors proximate to the current claim vector in a multidimensional vector space based at least in part on a distance between the current claim vector and the one or more claim vectors in the multidimensional vector space, the claim vector database storing a plurality of claim vectors corresponding to a plurality of prior claims, each claim vector comprising a multidimensional data structure representing semantic content in a corresponding prior claim;
query a provision vector database to identify one or more provision vectors corresponding to the plurality of codes, the provision vector database storing a plurality of provision vectors corresponding to a plurality of segments of one more provisioning structures, each provision vector comprising a multidimensional data structure representing semantic content in a corresponding segment;
apply a predictive large language model (LLM) to the current claim vector, the one or more claim vectors, the one or more provision vectors, and a schedule corresponding to the one or more provisioning structures to generate an optimal coding for the current claim record based at least in part on one or more optimization criteria; and
transform the current claim record based at least in part on the determined optimal coding.

18. The at least one non-transitory computer-readable medium of claim 17, wherein the current claim record is generated by:
receiving a visitation record comprising unstructured data;
encoding the visitation record as a visitation vector, the visitation vector comprising a multidimensional data structure representing semantic content in the visitation record;
querying a guideline vector database to identify one or more guideline vectors corresponding to the unstructured data, the guideline vector database storing a plurality of guideline vectors corresponding to a plurality of segments of one or more coding guideline structures, each guideline vector comprising a multidimensional data structure representing semantic content in a corresponding segment; and
applying the predictive large language model (LLM) to the visitation record and the one or more guideline vectors to generate the current claim record including the plurality of codes.

19. The at least one non-transitory computer-readable medium of claim 17, further storing computer-readable instructions that, when executed by at least one of the one or more computing devices, cause at least one of the one or more computing devices to:
transmit a representation of the multidimensional vector space in a user interface;
transmit a representation of current claim vector in the representation of the multidimensional vector space; and
transmit one or more representations of the one or more claim vectors proximate to the current claim vector in the representation of the multidimensional vector space.

20. The at least one non-transitory computer-readable medium of claim 19, further storing computer-readable instructions that, when executed by at least one of the one or more computing devices, cause at least one of the one or more computing devices to:
determine one or more outcome values for one or more prior claims corresponding to the one or more claim vectors proximate to the current claim vector, each outcome value indicating an outcome of a prior claim;
determine an approval probability value for the current claim record based at least in part on the one or more outcome values, the one or more claim vectors, and the current claim vector; and
transmit the approval probability value in the user interface.

21. The at least one non-transitory computer-readable medium of claim 17, wherein the provision vector database is generated by:
receiving one more provisioning structures;
segmenting the one or more provisioning structures to generate a plurality of segments; and
encoding the plurality of segments to generate the plurality of provision vectors, each provision vector comprising a multidimensional data structure representing semantic content in a corresponding segment.

22. The at least one non-transitory computer-readable medium of claim 21, wherein the schedule corresponding to the one or more provisioning structures is generated by:
parsing the one or more provisioning structures to identify a plurality of provisioning structure codes and a plurality of provisions;
identifying one or more provisions in the plurality of provisions related to each provisioning structure code in the plurality of provisioning structure codes; and
generating the schedule based at least in part on the plurality of provisioning structure codes and the one or more provisions related to each provisioning structure code.

23. The at least one non-transitory computer-readable medium of claim 17, wherein the optimization criteria comprises one or more of:
a predicted probability of approval of the current claim record;
an overall revenue resulting from the current claim record; or
a predicted response time for the current claim record.

24. The at least one non-transitory computer-readable medium of claim 17, wherein transforming the current claim record based at least in part on the determined optimal coding comprises one or more of:
replacing at least one code in the plurality of codes with at least one alternate code;
removing at least one code in the plurality of codes;
adding at least one new code to the plurality of codes;
changing an ordering of two or more codes in the plurality of codes; or
modifying a description associated with at least one code in the plurality of codes.
